(19) United States
(12) Patent Application Publication
(10) Pub. No.: US 2023/0223141 A1
(43) Pub. Date: Jul. 13, 2023

(54) INSTRUMENT AND SUPPLYING APPARATUS OR APPARATUS GROUP AND METHOD FOR OPERATION THEREOF

(71) Applicant: Erbe Elektromedizin GmbH, Tuebingen (DE)

(72) Inventors: Daniel Lang, Jettingen (DE); Juergen Fink-Metz, Reichenbach (DE); Andreas Guth, Gomaringen (DE)

(73) Assignee: Erbe Elektromedizin GmbH, Tuebingen (DE)

(21) Appl. No.: 18/093,169

(22) Filed: Jan. 4, 2023

(30) Foreign Application Priority Data
Jan. 13, 2022 (EP) 22151433.4

(51) Int. Cl. G16H 40/63; A61B 90/90

(52) U.S. Cl. CPC G16H 40/63; A61B 90/90; A61B 2562/08

(57) ABSTRACT

Instruments having plugs with data carriers containing codes identifiable as being correlated. The codes can be individual identifiers that are individually provided to the plugs. If plugs identical in construction of instruments of identical types are connected with apparatus or an apparatus group, e.g., a first plug of a used instrument and a second and third plug of a new instrument that replace the used instrument, the three inserted plugs are determined as not being part of the same instrument and an operation of the apparatus and thus the instruments is blocked.

### CROSS-REFERENCE TO RELATED APPLICATION

This application claims priority to European Patent Application No. 22151433.4, filed Jan. 13, 2022, the entirety of which is incorporated by reference herein.

Embodiments of the invention include devices for medical treatment of a human or animal patient. In addition, embodiments refer to an apparatus or apparatus group for supply of such instruments. Embodiments also refer to a device consisting of such an instrument and such an apparatus or apparatus group for treatment of human or animal patients as well as methods for operation.

For supply of surgical instruments with operating media, medical apparatuses are in use that provide, for example, high frequency electrical voltage, other media required for the treatment process, such as gases, e.g. argon, cryofluids or also vacuum for suction of fluids.

To guarantee in the practical daily routine that matching apparatuses and instruments are connected with one another and misconnections are avoided as far as possible, EP 2 656 806 B1, hereby incorporated by reference, provides a data interface at the plug in order to transmit information from the plug to the apparatus about the actual operating condition of the instrument, fault conditions, information about the instrument type, etc. The data interface is realized as one-wire interface (quasi industry standard of company Maxim Integrated) having a four pole contact, whereby also other standards or contact types can be considered, such as UART/RS-232, SPI, I2C, USB, Mini-USB or Micro-SD interface.

In addition, a surgical apparatus connector system is known from DE 10 2007 061 483 A1, hereby incorporated by reference, in which a coding plug is provided on the connector, the coding plug transmitting information to the apparatus based on which the apparatus specifically opens or closes switches for supply of the instrument with voltage or current.

The solutions existing so far assume that the instrument has only one single plug to be connected with an apparatus. However, instruments with multiple plugs exist. If instruments are changed during operation, which may occur due to wear or as result from the treatment process, all of the plugs of the former instrument have to be disconnected and all plugs of the new instrument have to be correctly connected again to one or more respective supplying apparatuses. Thereby, confusions must not result.

Starting therefrom it is the object of embodiments of the invention to provide a concept with which a wrong connection of a medical or surgical instrument to an apparatus or multiple apparatuses can be avoided with high reliability.

The instrument for medical treatment of a human or animal patient according to the invention comprises a first plug that is configured for connection with a supplying apparatus as well as at least one additional plug that is also configured for connection with the same or with another apparatus. For example, the first apparatus can supply an operating medium, such as cryo-fluid or the like, to the instrument. The socket configured at the same or another apparatus for the additional plug can be provided, for example, for supply of the instrument with electrical voltage, e.g. high frequency voltage for tissue coagulation or for other purposes.

Independent of how many plugs are provided on the instrument and independent of whether the assigned sockets are configured on one single or on multiple apparatuses, the plugs comprise data carriers that can be read by the apparatus on which the assigned socket is arranged. The data carriers of the plugs thereby contain identifiers that are coordinated with one another, so that the connected apparatus or the multiple connected apparatus are able to determine whether the plugs are part of one and the same instrument. Such an instrument allows the apparatus to check whether all plugs that occupy the respective sockets of the apparatus or multiple apparatus are part of one and the same instrument, i.e. whether they have to be considered as being correlated.

The check of correlation of the plugs by the apparatus avoids an otherwise possible severe error case that may particularly happen when changing instruments during an operation. If actually instruments shall be exchanged under time pressure and an obsolete instrument has to be disconnected from the multiple apparatus and a new instrument has to be connected to the multiple apparatus, it could happen that one or two plugs of the obsolete instrument still remain at the respective socket and only one or two plugs of the new instrument are connected. If in such a case a supposed correctly connected new instrument would be put into operation, it cannot function correctly and would result in damages in the worst case. In the best case, it would be inoperative, which disturbs the operation process. Such events are avoided by embodiments according to the invention, because the possibility is provided that the supplying apparatus or the apparatus system consisting of multiple apparatuses determines and quickly and clearly signals the connection of non-correlated plugs, i.e. plugs of different instruments, as error case.

In order to allow such a correlation check, multiple possibilities exist. For example, the first plug can comprise copies of the identifiers of the additional plugs as identifier. Alternatively, the first plug can comprise a calculation value formed from the identifiers of the additional plugs, e.g. a check sum, a hash code or the like, as identifier. The identifier of a plug (or multiple plugs) can also contain additional information, such as an instrument type identifier or an own individual identifier.

In all of the plugs, particularly however in the first plug, the respective data carrier can consist of one or of multiple physically separately configured individual data carriers. This offers the possibility to provide all plugs with data carriers (read only memories) that carry a consecutive non-variably stored number as individual identifier. At least in the first plug an additional individual data carrier can be provided that contains a calculation value formed of the individual identifiers.

In the instrument according to the invention all of the plugs are characterized by the provided individual identifiers as being correlated and thus as being part of the same instrument, in that a stored value can be accessed in or on the instrument, e.g. in one of the plugs, that can be unambiguously derived from the individual identifiers of the individual plugs according to a suitable formation rule. The formation rule can be a check sum formation rule. The one apparatus/the multiple apparatuses can be configured to form a calculation value from the individual identifiers according to the same formation rule and to only start operation, if the calculation value corresponds to the stored value.

For realization of this concept different variants are possible. For example, in a first variant all plugs can have the same individual identifier so that an apparatus control has to check only whether all individual identifiers correspond.

In another variant the first plug contains the individual identifiers of the additional plugs. In doing so, the apparatus control can allow the operation of the apparatus or the apparatus system, if all individual identifiers that are stored in the first plug and that have been read therefrom are retrieved and read on the sockets of the additional plugs.

In a third variant the first plug contains a calculation value formed from the individual identifiers of the other plugs. The apparatus control forms a calculation value according to the same calculation rule from the individual identifier read from the sockets of the additional plugs and allows operation of the apparatus or apparatus system, if this calculation value corresponds with the calculation value stored in the first plug.

The calculation value can also be a constant that is equal for all of the instruments. For example, the calculation value can be the sum of the identifiers of the plugs or another calculation value formed from the identifiers of the plugs. This provides the possibility to store the calculation value not in one of the plugs, but only in the apparatus and indeed independent of the number of the plugs (two, three or more) that are part of the system.

In another variant each plug contains an individual identifier that is considered during formation of the calculation value. Also in this case the apparatus control follows the scheme mentioned above, whereby, however, it now considers the individual identifiers of all plugs.

According to any of the principles mentioned above, the identifier of the first plug and the individual identifiers of the additional plugs can be considered as coordinated individual identifiers.

Preferably each plug comprises a communication device capable of carrying out a data exchange with the apparatus or apparatus system at the respective socket provided for the plug. The communication device can be a contactless communication module or a contact module. For example, the contactless communication module can be a NFC-chip or an optical module. For example, the contact module can be realized by a contact group, such as the contacts of a USB interface or the like.

Embodiments of the invention are also realized in an apparatus or apparatus group with which an instrument having multiple plugs is to be supplied. For example, an apparatus in accordance with an embodiment described herein, comprises sockets that are configured to connect a first plug as well as a second plug. Further, communication modules are arranged on the sockets that are configured to establish a data connection with the communication modules of the plugs. By way of the communication modules, the control device of the apparatus or apparatus group can read the individual identifiers of the plugs and can check them for consistency. The consistency check can be carried out according to any of the principles mentioned above. Preferably a consistency check is carried out based on the determination of a calculation value from the individual identifiers, wherein this calculation value must correspond with a calculation value that is preferably stored in the first plug.

The control device can be realized by the individual control devices of the multiple apparatuses of an apparatus system that are connected with one another for data exchange, e.g., by a communication bus.

The control device is configured to check the correlation of the plugs (consistency check). In doing so, it is checked whether the plugs are part of one and the same instrument. The operation of the apparatus or apparatus group is only allowed by the control device if the consistency check is passed, i.e., if the plugs have been determined as being correlated.

Further details of advantageous embodiments of the concept according to the invention can be derived from the claims, as well as the drawing and the respective description. In the drawing embodiments of the invention are illustrated. The drawings show:

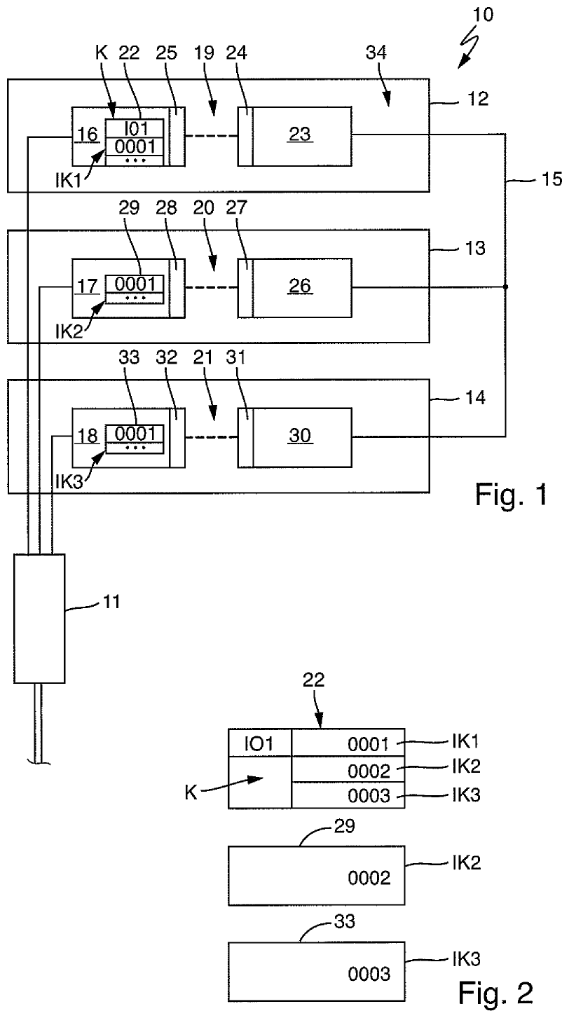

FIG. 1 illustrates a schematic overview of an apparatus system with a connected instrument in accordance with an embodiment of the invention;

FIGS. 2-5 illustrate different embodiments of data structures of plugs of the instrument in accordance with embodiments described herein.

A device 10 for treatment of human or animal patients is illustrated in FIG. 1. This device 10 comprises at least one medical or surgical instrument 11 and one or more apparatuses 12, 13, 14, to which it is connected. The apparatuses 12-14 are connected to one another by a bus 15 and therefore form an apparatus system. The apparatus system serves for supply of instrument 11 with the required operating media. For this purpose, instrument 11 is connected to the apparatus 12, 13, 14 by multiple plugs, e.g., a first plug 16 as well as at least one additional plug 17, 18. The apparatus 12, 13, 14 comprise respective sockets 19, 20, 21 for this purpose at which the plugs 16, 17, 18 receive the associated operating media. The plugs 16, 17, 18 are connected by lines with the instrument 11 to supply the operating media to the instrument 11.

The first plug 16 comprises a data carrier 22 that consists of one or more memory chips. On the data carrier 22, an identifier K is stored that can be read by an apparatus control 23. For this purpose, a communication module 24 is provided on the socket 19 on the side of apparatus 12. A communication module 25 matching thereto is comprised by plug 16, the communication module 25 being able to establish a data connection with communication module 24. The data connection between both communication modules 24, 25 is illustrated in FIG. 1 by a dashed line.

In similar manner the apparatus 13 comprises an apparatus control 26 having a communication module 27. It is configured to connect with a communication module 28 of plug 17. The communication module 28 is connected to a data carrier 29 on which an individual identifier IK2 is stored. Via the data connection between the communication modules 27, 28 the individual identifier IK2 can be read by apparatus control 26.

In accordance therewith apparatus 14 comprises an apparatus control 30 that can establish a data connection with a communication module 32 of plug 18 by a communication module 31. The communication module 32 is connected to a data carrier 33 of plug 18 on which an individual identifier IK3 can be stored.

In the extremely simplified depicted embodiment, the identifier K stored on the data carrier 22 of plug 16 consists of an instrument type designation, here by way of example I01, as well as in addition of an individual identifier IK1, here the number 0001. In the additional plugs 17 and 18 the data carrier 29 and 33 is respectively provided with the individual identifier IK2 and IK3 that are here also the number 0001 in the simplified example. According to this concept, the correlated plugs 16, 17, 18 of one and the same instrument 11 have corresponding individual identifiers IK1=IK2=IK3.

If such an instrument 11 is connected to the apparatus system consisting of apparatus 12, 13, 14, the control device 34 consisting of the apparatus controls 23, 26, 30 checks whether the individual identifiers IK1, IK2 and IK3 of plugs 16, 17, 18 correspond. If yes, the control device 34, shown as part of apparatus 12 for exemplary purposes only, allows the operation of apparatus 12, 13, 14.

FIG. 2 illustrates a modified embodiment in which only the data carriers 22, 29 and 33 of plugs 16, 17, 18 are illustrated for sake of simplification. Again, on the data carrier 22 the instrument type, here by way of example I01, is stored in the context of the stored identifier K. In addition, the data carrier 22 can contain the individual identifier IK1 of the first plug 16. The data carrier 22 contains the individual identifiers IK2 and IK3; the data carriers 29 and 30 of additional plugs 17 and 18 contain individual identifiers as well. In such a system, the control device 34 checks prior to allowing operation of apparatus 12-14 whether the sockets 18 and 19 have the correct identifiers IK2 and IK3 and can be read from the first plug 16 on socket 19. If this is the case, the control device is configured to allow operation. It is in addition configured to block operation as long as this is not the case.

In the embodiments mentioned above, data carriers 22, 29, 33 are required on the plugs 16, 17, 18 that are provided individually and coordinated with one another and that are provided with data coordinated with one another. It is, however, also possible to provide at least the data carriers 29, 33 with arbitrary or randomly defined individual identifiers IK2 and IK3 or to use data carriers 29, 33 provided in this manner. In doing so, numbering chips taken from a series production can be used on which consecutive numbers are non-variably stored. When using the embodiment according to FIG. 2, then the individual identifiers IK2, IK3, as they randomly result from the current production, for example, are copied on the data carrier 22. The operation of the system is carried out according to the description above.

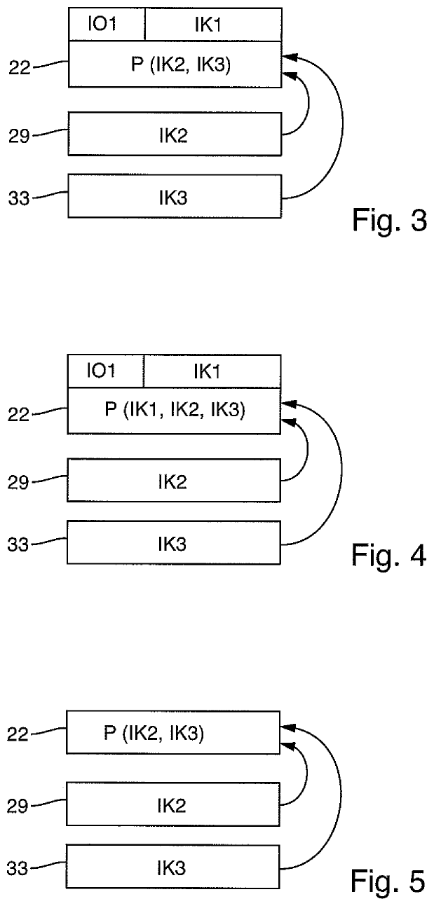

Instead of copies of individual identifiers IK2, IK3 it is in addition possible to only store a calculation value formed therefrom on data carrier 22 of plug 16, as illustrated in FIG. 3. The data carrier 22 there again contains an instrument identifier I01 and at least optionally an own first individual identifier IK1. The data carriers 29, 33 contain in turn individual identifiers IK2, IK3 that can be different from the individual identifier IK1. Particularly, they can also be different from one another. From the two individual identifiers IK2, IK3 of plugs 17 and 18 the calculation value P(IK2, IK3) is calculated during provision of first plug 16. For example, this calculation value can be a check sum, e.g. a CRC check sum, hash value or the like.

In operation, after insertion of first plug 16, the control device 34 checks whether additional plugs are inserted in the additional sockets 20, 21 and if this is the case, whether the readable individual identifiers IK2 and IK3 from the plugs 17 and 18 used there result in the check sum P(IK2, IK3) that is readable from first plug 16. If this is the case, the operation of apparatus 12-14 is allowed.

Largely similar is the embodiment according to FIG. 4 in which the check sum P(IK1, IK2, IK3) is formed in addition from the first individual identifier IK1 of first plug 16. Apart therefrom, the method operates as described above in relation to FIG. 3.

A further configuration is apparent from FIG. 5 in which data carriers 29, 30 of additional plugs 17, 18 contain the individual identifiers IK2 and IK3 as in all of the previous examples. The individual identifier IK1 stored on data carrier 22 of first plug 16 and includes only a check sum P(IK2, IK3). The consistency check whether the three plugs 16, 17, 18 connected with the apparatus 12, 13, 14 are part of one and the same instrument 11 is also reliably possible according to this simplified embodiment.

In all embodiments described above the instrument identifier I01 serves for identification for the instrument type. The apparatus 12, 13, 14 can be configured to adjust their operating parameters or ranges for the operating parameters according to the instrument type. In any embodiment presented here the instrument type identifier I01 can also be omitted, if it is not desired or not necessary.

An instrument 11 according to the invention comprises plugs 16, 17, 18 with data carriers 22, 29, 33 that contain codes identifiable as being correlated. Such codes can be individual identifiers IK1, IK2, IK3 that are individually provided to the plugs. If plugs identical in construction of instruments of identical types are connected with apparatus or an apparatus group, e.g., a first plug of a used up instrument and a second and third plug of a new instrument that shall replace the used up instrument, the three inserted plugs are determined as not being part of the same instrument and an operation of the apparatus 12, 13, 14 and thus the use of the instruments is blocked. In doing so, the operation safety is remarkably increased when using the embodiments described herein.

1. An instrument for medical treatment of a human or animal patient, comprising:
a first plug configured for connection with an apparatus;
a second plug configured for connection with the apparatus or another apparatus;
wherein the first plug comprises a data carrier having a first identifier; and
wherein the second plug comprises a data carrier having a second identifier different from the first identifier.

2. The instrument according to claim 1, characterized in that the first identifier comprises an instrument identifier as well as an individual identifier.

3. The instrument according to claim 1, characterized in that each of first and second plugs comprise data carriers with individual identifiers that are coordinated with one another.

4. The instrument according to claim 1, characterized in that the data carrier of the first plug comprises the individual identifiers of the second plug or a calculation parameter based thereon.

5. The instrument according to claim 1, characterized in that the data carrier of the first plug comprises a hash code or a check identifier of individual identifiers of the second plug.

6. The instrument according to claim 1, characterized in that the data carrier of the first plug comprises a hash code or a check identifier of the individual identifiers of the second plug.

7. The instrument according to claim 1, characterized in that the first and second plugs are respectively connected with a communication device configured to transmit the individual identifier to an apparatus to be connected.

8. The instrument according to claim 7, characterized in that the communication means is a radio module.

9. The instrument according to claim 7, characterized in that the communication means is a contact module.

10. The instrument according to claim 1, characterized in that the first plug and the second plug are configured for transmission of different media.

11. An apparatus or apparatus group for supply of an instrument according to claim 1, further comprising
sockets that are configured for insertion of the first and second plugs and detection of the first and second identifiers, and
a control device that is configured to check the correlation of the plugs.

12. The apparatus or apparatus group according to claim 11, characterized in that the control device is configured to check the correlation of the first and second plugs based on the first and second identifiers of the first and second plugs.

13. The apparatus or apparatus group according to claim 11, characterized in that the control device is configured to only allow the operation of the instrument, if the first and second plugs of the instrument connected with the apparatus or the apparatus group are determined as being correlated.

14. A device for treatment of a human or animal patient comprising the apparatus or apparatus group according to claim 11.

15. A method for operation of a device according to claim 14, characterized in that after the connection of the first and second plugs with the sockets the first and second identifiers stored in the first and second plugs are correlated to determine whether the plugs are part of the same instrument and the operation of the instrument is only allowed, if the plugs are part of the same instrument.
